(19) United States
(12) Patent Application Publication
(10) Pub. No.: US 2024/0409495 A1
(43) Pub. Date: Dec. 12, 2024

(54) PROCESS FOR THE PREPARATION OF 2,7-DIHYDROXY-9-FLUORENONE USEFUL FOR THE SYNTHESIS OF TILORONE AND ITS SALTS

(71) Applicant: Council of Scientific & Industrial Research, New Delhi (IN)

(72) Inventors: Parasuraman JAISANKAR, Kolkata (IN); Indubhusan DEB, Kolkata (IN); Pinaki BHATTACHARJEE, Kolkata (IN)

(21) Appl. No.: 18/702,639

(22) PCT Filed: Oct. 19, 2022
(86) PCT No.: PCT/IN2022/050931

§ 371 Date: Apr. 18, 2024

(30) Foreign Application Priority Data
Oct. 20, 2021 (IN) 202111047809

(51) Int. Cl. C07C 213/02; C07C 45/27; C07C 45/64; C07C 201/12; C07C 221/00

(52) U.S. Cl. CPC C07C 213/02; C07C 45/27; C07C 45/64; C07C 201/12; C07C 221/00

(57) ABSTRACT

Methods involving preparation of building blocks of 2,7-dihydroxy-9-fluorenone toward the synthesis of tilorone dihydrochloride salt and other salts (bromide, iodide, fluoride, citrate, oxalate, maleate, phosphate, tartrate, triflate, trifluoroacetate, tetrafluoroborate) of tilorone, and an efficient, safe, cost effective method for the preparation of 2,7-bis-[2-(diethylamino)ethoxy]-fluorenone-9 and its various salts are developed. The methods involve oxygenation of fluorene, nitration of fluorenone, followed by reduction and diazotization toward the formation of 2,7-dihydroxy-9-fluorenone, which is the key intermediate for the formation of 2,7-bis-[2-(diethylamino)ethoxy]-9-fluorenone-dihydrochloride (Tilorone dihydrochloride) and other tilorone salts. The synthesis method has relatively simple operation, mild reaction conditions, high yield, and simple process with yields of 80-97%. Subsequent product purification of this method uses filtration and crystallization methods, avoiding the existing methods of column chromatography. Therefore, the research of its synthetic method being with a wide range of applications from the drug development and material synthesis point of view.

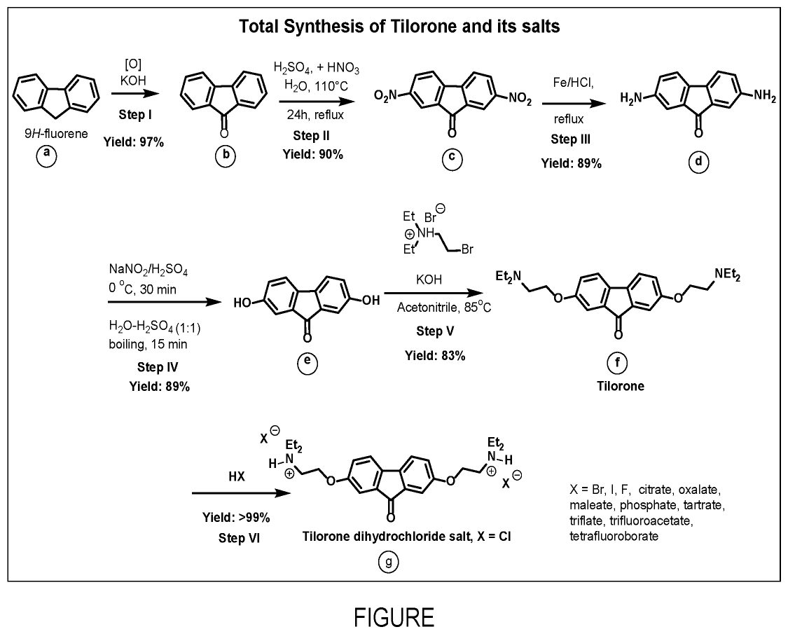

### CROSS REFERENCES TO RELATED APPLICATIONS

This application is a national-stage application under 35 U.S.C. § 371 of International Application No. PCT/IN2022/050931, filed Oct. 19, 2022, which International Application claims benefit of priority to Indian Patent Application No. 202111047809, filed Oct. 20, 2021.

### TECHNICAL FIELD

The present application relates to a multistep method for the preparation of 2,7-dihydroxy-9-fluorenone. Particularly, the present application relates to a method for the preparation of 2,7-dihydroxy-9-fluorenone useful for the synthesis of tilorone and its salts of selected from the group consisting of bromide, iodide, fluoride, citrate, oxalate, maleate, phosphate, tartrate, triflate, trifluoroacetate, tetrafluoroborate. More particularly, present application relates to a method for the preparation of tilorone dihydrochloride salt which is an orally active interferon inducing agent.

### BACKGROUND

As one of the separated product of coal tar, fluorenes output is large, cheap, and its derivatives are very important fine chemicals intermediate and of many uses. (2,7-bis[2-(diethylamino)ethoxy]-9H-fluoren-9-one) is one of the fluorene derivatives. A promising drug tilorone dihydrochloride (2,7-bis[2-(diethylamino)ethoxy]-9H-fluoren-9-one) is a small molecule (410.549 Da) that is orally bioavailable interferon inducer is synthesized here in a systematic fashion from fluorene. The well-known drug Tilorone has a broad-spectrum antiviral activity. It was reported that Tilorone's antiviral activity is acting through an IFN-related innate immunity pathway. In vivo efficacy studies from the literature showed possible uses of tilorone against a broad array of infections including Middle East Respiratory Syndrome, Chikungunya, Ebola and Marburg and more recently against human coronaviruses including MERS-CoV which highlights that this old drug may be repurposed against the new viruses that are responsible for the pandemic situation right now. To support urgent research to combat the ongoing outbreak of COVID-19, caused by the novel coronavirus SARS-CoV-2, our focus is to synthesis Tilorone in a novel and environment and industry-friendly manner. Even, the compounds in the intermediate steps are widely used in, preparation of dyestuff, plastics, medicine and electroluminescent material etc. Therefore, the research of its synthetic method has a wide range of applications from drug development and material synthesis point of view.

The present disclosure process provides an efficient, safe, cost effective way to prepare 2,7 dihydroxy fluorenone (II) towards the total synthesis of Tilorone dihydrochloride (II) and other Tilorone salt forms. Compound (I) is the intermediate of the final compound (II) which is known as an orally active interferon inducer.

Literature precedence supports that, the target compound Tilorone was prepared from different perspectives. According to the method of Andrews (J. Med. Chem. 1974, 17, 8, 882-886) towards the synthesis of Tilorone, 4,4-dihydroxy-[1,1-biphenyl]-2-carboxylic acid was synthesized as one of the intermediate step but further it was found that compound 4,4-dihydroxy-[1,1-biphenyl]-2-carboxylic acid could not be fully mixed with ZnCl₂ and converted into 2,7-dihydroxy-9-H-Fluorenone at 200° C., especially when the reaction scale was large. After that Burke et al. showed a synthetic pathway to Tilorone Hydrochloride (Synthetic Communications, 1976, 6 (5), 371-376) but they did not clearly mention the actual yields and conditions for the intermediate steps associated for the synthesis. Further, the total synthesis of Tilorone was prepared by using anew method of multi-step oxidation method (J Material Chem. 2008, 18, 3361-3365; Chinese patent, Patent no: CN102424651A, Russian Patent, Patent no: RU2444512C1). Due to the shortcomings of high cost (Tetrahedron, 2019, 75(2), 236-245, Organic Letters, 2017, 19(5), 1140-1143), dangerous operation, high-temperature reactions, complexity and large amount of solvent, it is difficult to obtain satisfactory results in industrial preparation.

The previously reported procedure involves tedious steps and each step needs to be purified by column chromatography, which is expensive and time consuming for commercial operation.

A main object of the present disclosure is to provide a process for the preparation of 2,7-dihydroxy-9-fluorenone of formula (e).

Another object of the present disclosure is to provide a process for the preparation of 2,7-dihydroxy-9-fluorenone useful in the manufacture of tilorone of formula (f) and its salts of formula (g) especially, Tilorone dihydrochloride ((2,7-bis[2-(diethylamino)ethoxy]-9H-fluoren-9-one) in high yield.

Yet another object of the present disclosure is to provide synthetic methodology towards the synthesis of interferon inducer tilorone drug ((2,7-bis[2-(diethylamino)ethoxy]-9H-fluoren-9-one).

Yet another object of the present disclosure to provide (2,7-bis[2-(diethylamino)ethoxy]-9H-fluoren-9-one) in high chemical yield.

Yet another object of the present disclosure is to provide a commercially viable process for the synthesis of 2,7-dihydroxyfluorenone towards 2,7-bis[2-(diethylamino)ethoxy]-9H-fluoren-9-one synthesis.

Yet another object of the present disclosure is to provide each steps user friendly and operationally benign towards the synthesis of (2,7-bis[2-(diethylamino)ethoxy]-9H-fluoren-9-one).

Yet another object of the present disclosure is to provide novel methodology with a cheaper starting material.

### SUMMARY OF THE INVENTION

Accordingly, the present disclosure provides a process for the preparation of 2,7-dihydroxyfluorenone of formula (e) comprising the steps of:

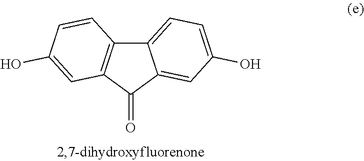 [SMILES: O=C1c2cc(O)ccc2-c2ccc(O)cc21]

i. reacting fluorene of formula (a), organic solvent and mineral alkali at room temperature in the range of 20 to 30° C. followed by filtering, mixing ice, blending and again filtering to obtain Fluorenone of formula (b);

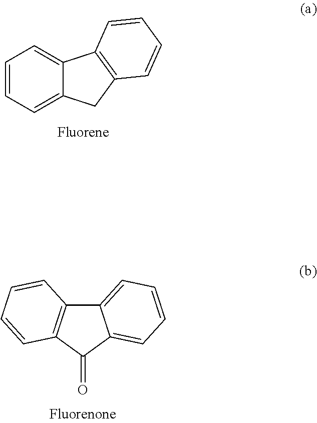

ii. refluxing Fluorenone of formula (b) as obtained in step (i), water and mixed acid of concentrated sulfuric acid and concentrated nitric acid in 1:1 ratio followed by filtering to obtain 2,7-dinitro-9-fluorenone of formula (c);

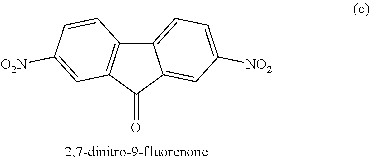 [SMILES: O=C1c2cc([N+](=O)[O-])ccc2-c2ccc([N+](=O)[O-])cc21]

iii. dissolving 2,7-dinitrobenzene-9-fluorenone of formula (c) as obtained in step (ii) in water alcohol solution followed by adding iron powder and concentrated hydrochloric acid under reflux condition followed by suction filtration to obtain 2,7-diamino-9-fluorenone of formula (d);

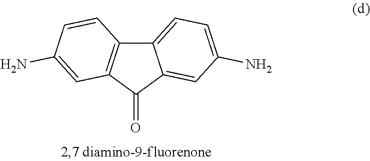 [SMILES: Nc1ccc2c(c1)C(=O)c1cc(N)ccc1-2]

iv. dissolving 2,7 diamino-9-fluorenone of formula (d) as obtained in step (iii) in water and mineral acid having ratio 1:1.33 with addition of sodium nitrite, cooling at temperature in the range of −10° C. to 0° C. followed by refluxing with mineral acid and water and filtering to obtain 2,7-Dihydroxy-9-fluorenone of formula (e).

In an embodiment, in step (i), mineral alkali used is sodium hydroxide or potassium hydroxide with 1.2 equivalent.

In another embodiment, the alcohol used in step (iii) is selected from the group consisting of ethanol, methanol, isopropanol preferably ethanol.

In yet another embodiment, in step (iv), the mineral acid used is selected from the group consisting of concentrated hydrochloric acid, concentrated sulfuric acid or hydrobromic acid preferably sulfuric acid.

In yet another embodiment, in step (iv), the mineral acid used for refluxing is sulfuric acid.

In yet another embodiment, 2,7-dihydroxy-9-fluorenone is useful for the synthesis of tilorone of formula (f) and its salts of formula (g) useful as immunomodulator and as broad-spectrum of antiviral agent and the said process comprising the steps of:

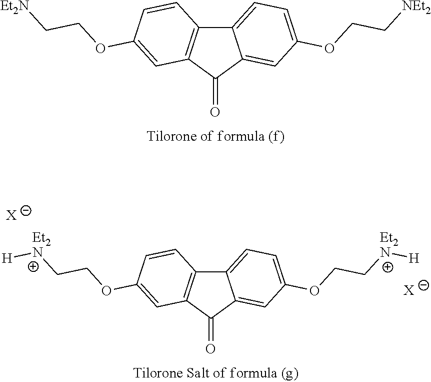

wherein X=Cl, Br, I, F, citrate, oxalate, maleate, phosphate, tartrate, triflate, trifluoroacetate, tetrafluoroborate;
i. dissolving 2,7-dihydroxyfluorenone in organic solvent and adding 2-Bromo-N,N-diethylethylamine hydrobromide and mineral alkali followed by refluxing at temperature in the range of 75° C. to 160° C. preferably 85-110° C. to afford 2,7-Bis[2-(diethylamino)ethoxy]-9H-fluoren-9-one [Tilorone] of formula (f) followed by acidifying with acid to afford the Tilorone salt of formula (g).

In yet another embodiment, the organic solvent is selected from the group consisting of tetrahydrofuran, 1,4-dioxan, toluene, benzene, acetonitrile, dimethyl formamide (DMF), dimethyl sulfoxide, ethyl acetate or acetone preferably acetonitrile.

In yet another embodiment, the mineral alkali used is KOH.

In yet another embodiment, the tilorone salt is selected from the group consisting of chloride, bromide, iodide, fluoride, citrate, oxalate, maleate, phosphate, tartrate, triflate, trifluoroacetate, tetrafluoroborate.

In yet another embodiment, tilorone dihydrochloride salt is an orally active interferon inducing agent.

### BRIEF DESCRIPTION OF THE DRAWINGS

The FIGURE represents total synthesis of Tilorone and its salt.

### DETAILED DESCRIPTION

The present disclosure provides a method for the synthesis of 2,7-dihydroxy-9-fluorenone of formula (e) towards the synthesis of 2,7-bis[2-(diethylamino)ethoxy]-9H-fluoren-9-one (f) and its salt of formula (g) from fluorene of formula (a).

Fluorene can be made by utilizing the following procedure where DMA was added to a vial containing o-dibromobenzene, 2-methylarylboronic acid, K₂CO₃, palladium (II) acetate, tricyclohexylphosphine, and trimethyl acetic acid. After the reaction mixture had been stirred at 140-150° C. for 5 h, the mixture was cooled down to room temperature. The reaction mixture was quenched by adding water. The mixture was extracted with CH₂Cl₂ and solvent was evaporated to dryness which was further column purified to afford the desired fluorene of formula (a) in excellent yield (90%).

The key step is to react fluorene with DMF organic solvents and inorganic base such as KOH at room temperature in oxygen atmosphere. After the reaction is completed, neutralization with acid was done and, suction filtration to obtain 9H-fluorenone.

The organic solvent used is DMF.

The reaction of 9H-fluorenone with concentrated sulfuric acid and concentrated nitric acid at refluxing condition to obtain 2,7-dinitrofluorenone. After completion of the reaction suction and filtration performed to get yellow solid.

2,7-dinitrofluorenone dissolved in alcohol solution, added iron powder and concentrated HCl under reflux condition. When the reaction is completed, filter by suction to obtain 2,7-diaminofluorenone. Which further diazotised upon treatment of Sodium nitrite solution and inorganic acid and subsequently refluxed with 50% inorganic acid [mineral acid] solution to afford 2,7-dihydroxy-9-fluorenone. Alkylation to the 2,7-dihydroxy-9-fluorenone with 2-bromo-N,N-diethylethylamine hydrobromide in acetonitrile under refluxing condition afforded desired Tilorone. This was acidified to obtain the Tilorone dihydrochloride salt and other salts of tilorone.

In the diazotization step Sulfuric, Hydrochloric, Tetrafluoroboric acid and hydrobromic acid were used.

### EXAMPLES

The following examples are given by way of illustration and therefore should not be construed to limit the scope of the application or the claims.

### Example 1: Preparation of Fluorene (Formula a)

DMA (2 mL) was added to a vial containing o-dibromobenzene (0.5 mmol), 2-methylarylboronic acid (0.55 mmol), K₂CO₃ (3 mmol), palladium (II) acetate (0.015 mmol), tricyclohexylphosphine (0.03 mmol), and trimethylacetic acid (0.5 mmol). After the reaction mixture had been stirred at 140-150° C. for 5 h, the mixture was cooled down to room temperature. The reaction mixture was quenched by adding water. The mixture was extracted with CH₂Cl₂. The organic layer was washed with brine. After removal of the solvents the crude reaction mixture was purified by column chromatography on silica gel with hexanes as the eluent to afford the desired fluorene (1) in excellent yield (90%).

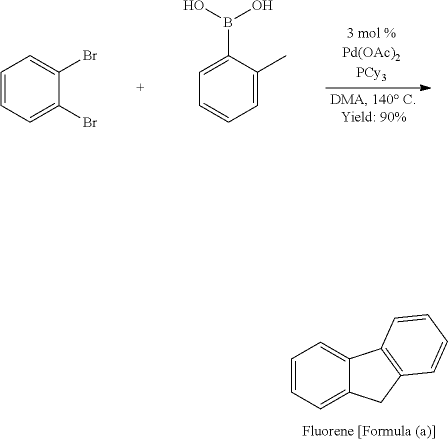

### Example 2: Step I: Preparation of 9-fluorenone (Formula b)

Into a 250 mL, round bottom flask equipped with an electromagnetic stirrer and an air condenser were charged 9H-fluorenes (1 gm), KOH (412 mg, 1.2 eqv) and DMF (15 mL), and stirred at room temperature (30° C.) under oxygen atmosphere (Oxygen purging). In a beaker containing ice this reaction mass was poured after completion of the reaction and then the neutralization was done with concentrated HCl by checking the pH with pH paper. Then precipitated was appeared and filtered through sintered funnel. The yellow precipitate was dried under reduced pressure. No need for column purification. Yield: 1.07 g (97%).

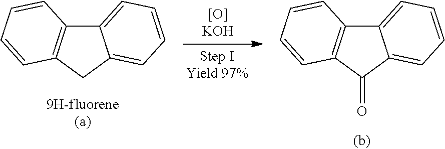

### Example 3: Step II: Preparation of 2,7 dinitro-9-fluorenone (Formula c)

In a 250 ml round bottom flask equipped with a mechanical stirrer, reflux condenser, add 2.5 gm of fluorenone, add 6 mL of water, start stirring, and heat the oil bath to 80° C., add the Mixed acid (concentrated nitric acid and concentrated sulfuric acid (1:1)=19 mL). After refluxing for 24 h at 120° C. Yellow cake like reaction mass was obtained which was filtered through sintered funnel by using ice cold water to obtain 2,7 dinitrofluorenone as yellow solid. No need for column purification. Yield: 3.38 g (90%)

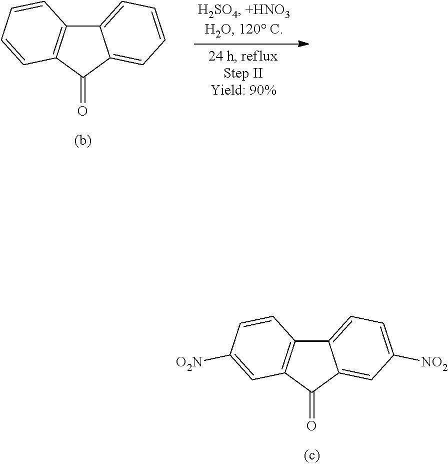 [SMILES: O=C1c2ccccc2-c2ccccc21 ; O=C1c2cc([N+](=O)[O-])ccc2-c2ccc([N+](=O)[O-])cc21]

### Example 4: Steps III & IV: Preparation of 2,7 dihydroxy-9-fluorenone (Formula e)

To a solution of 2,7-dinitrofluorenone (1.5 g) in ethyl alcohol water mixture (5:1) (60 mL), was added iron powder (9.3 g), and Concentrated HCl 38 ml under nitrogen atmosphere. The mixture was stirred for 24 h at the reflux temperature (110° C.). The pH of the mixture was made basic by addition of aqueous sodium hydroxide and finally was extracted with ethyl acetate. The organic layer (Violet coloured ethyl acetate layer) was dried over anhydrous sodium sulfate and evaporated to dryness to obtain 2,7-diaminofluorenone as brown coloured solid (Yield: 89%). Which was directly used for performing diazotization reaction without performing column purification.

2,7-diaminofluorenone (1 g) was suspended in 57 mL of water (ice bath) to which 76 mL of concentrated H₂SO₄ was added, at which point all of the solid dissolved. A solution of NaNO₂ (850 mg) in 38 mL of water was added dropwise over 5 min with stirring. Stirring was continued for a further 150 min, at which time the pale-yellow colour disappeared leaving a yellowish-brown coloured solution. This solution was added dropwise (over 15 min) to a boiling solution (110° C.) of 1:1 (v/v) concentrated H₂SO₄—H₂O (150 mL). After addition, the mixture was boiled for a further 15 min and then allowed to cool. The reaction mixture was extracted with ethylacetate, and the combined organic layers were extracted with NaOH solution to maintain just basic pH. Then the crude 2,7-dihydroxyfluorenone was extracted with ethylacetate by acidification (10% HCl) of the aqueous extract, and airdried.

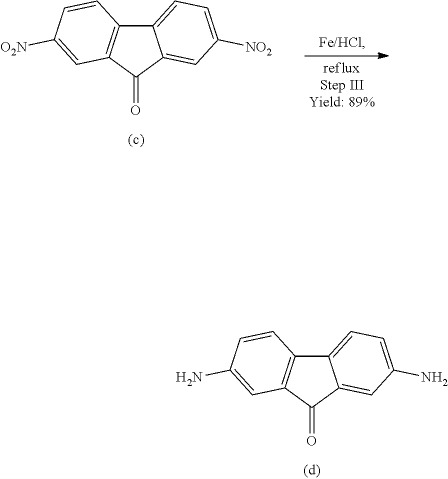 [SMILES: O=C1c2cc([N+](=O)[O-])ccc2-c2ccc([N+](=O)[O-])cc21 ; Nc1ccc2c(c1)C(=O)c1cc(N)ccc1-2]

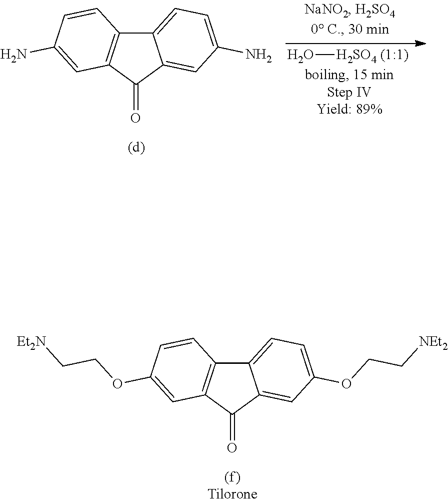

### Example 5: Step V & VI: Preparation of Tilorone dihydrochloride (Formula g)

250-mL round bottom flask equipped with a stir bar was charged with 2,7-dihydoxy-9H-fluorenone (1 g), 2-bromo-N, N-diethylethylamine hydrobromide (4.55 g) and KOH (2.26 g), followed by the addition of 15 mL acetonitrile and 3.2 mL distilled water. The reaction was then stirred vigorously and heated to reflux for 20 h. After cooling to room temperature, the reaction mixture was poured into water and extracted with Et₂O. The combined organic layers were dried over Na₂SO₄, and the solvent evaporated under reduced pressure. The resulting residue was dried thoroughly to get the desired product as an orange gummy solid (1.6 g, 83%). Which was dissolved in dry ether and added 2M ethereal HCl solution drop by drop to obtain Tilorone dihydrochloride salt with >99% yield (1.88 g) and the orange product was recrystallized with anhydrous ethanol.

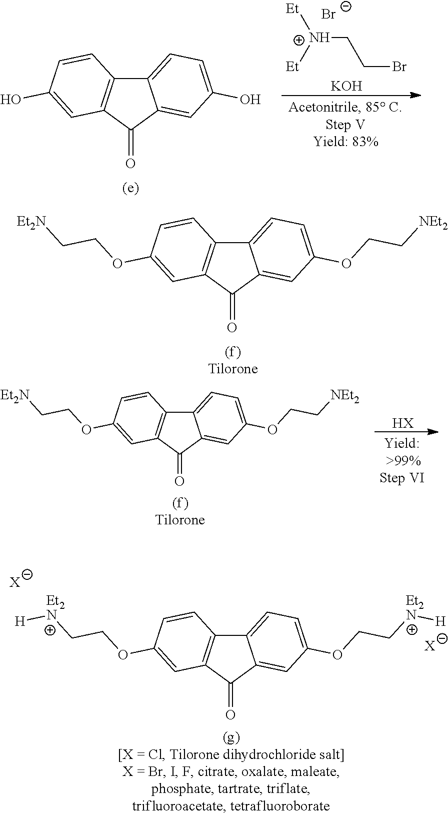

There is an urgent need of a better environmentally friendly and industrially viable methodology. Our established method provides a simple strategy for synthesizing Tilorone from fluorene with high yield, simple reaction operation, low cost and less environmental pollution. As one of the separation products of coal tar, fluorene has large output and low price. Its derivatives are widely used as important fine chemical intermediates. In order to improve the efficiency and practicability of this reaction, we found out the following useful method which provides a simple and feasible new method with low cost, high yield and suitable for industrial production.

The synthesis method of embodiments has relatively simple operation, mild reaction conditions, high yield and simple process with, yield 80-99%.

Subsequent product separation of this method uses filtration and crystallization methods, avoiding the existing methods that require a large amount of water washing and column separation, low yield and hazardous chemicals.

The new methodology is simple and feasible. It is not only suitable for small-scale laboratory preparation, but also suitable for large-scale production.

The technical problem to be solved is to overcome the existing synthetic method for preparing Tilorone using high temperature and large amount of solvent, complex Multi-step operation, high cost of the chemicals and difficult to industrialize and other defects. Embodiments herein produce tilorone (f) and its salt forms (g) with high yield, simple reaction operation, low cost and less environmental pollution.

The yields of other procedures were not clearly mentioned and not up to the mark. Therefore, the problems still existed, including inconvenient practice and the existence of heavy material clashing due to the high temperature.

1-10. (canceled)

11. A process for preparing 2,7-dihydroxy-9-fluorenone, the process comprising:
(i) reacting fluorene of formula (a): 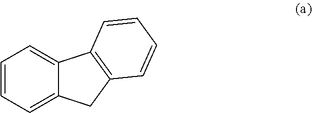 [SMILES: c1ccc2c(c1)Cc1ccccc1-2]
with an organic solvent and a mineral alkali at a temperature from 20° C. to 30° C., followed by filtering, mixing with ice, blending, and again filtering, to obtain fluorenone of formula (b): 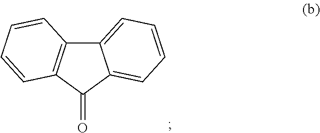 [SMILES: O=C1c2ccccc2-c2ccccc21]
(ii) refluxing the fluorenone obtained in (i) with water and a mixed acid comprising concentrated sulfuric acid and concentrated nitric acid in a 1:1 ratio, followed by filtering, to obtain 2,7-dinitro-9-fluorenone of formula (c): 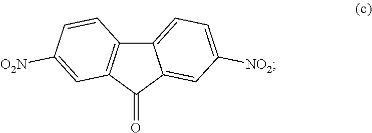 [SMILES: O=C1c2cc([N+](=O)[O-])ccc2-c2ccc([N+](=O)[O-])cc21]
(iii) dissolving the 2,7-dinitrobenzene-9-fluorenone obtained in (ii) in a water/alcohol solution, followed by adding iron powder and concentrated hydrochloric acid under reflux condition, followed by suction filtration, to obtain 2,7-diamino-9-fluorenone of formula (d): 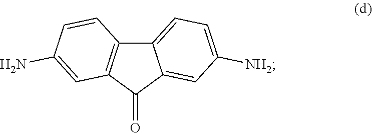 [SMILES: Nc1ccc2c(c1)C(=O)c1cc(N)ccc1-2]
(iv) dissolving the 2,7 diamino-9-fluorenone obtained in (iii) in water and a first mineral acid having ratio 1:1.33 with addition of sodium nitrite, cooling at a temperature from −10° C. to 0° C., followed by refluxing with a second mineral acid and water, and filtering, to obtain 2,7-dihydroxy-9-fluorenone of formula (e):

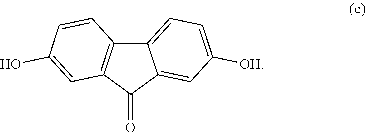 [SMILES: O=C1c2cc(O)ccc2-c2ccc(O)cc21]

12. The process according to claim 11, wherein in the mineral alkali of (i) is 1.2 equivalents of sodium hydroxide or potassium hydroxide.

13. The process according to claim 11, wherein the alcohol of (iii) is selected from the group consisting of ethanol, methanol, and isopropanol. preferably ethanol.

14. The process according to claim 11, wherein the alcohol of (iii) is ethanol.

15. The process according to claim 11, wherein the first mineral acid of (iv) is selected from the group consisting of concentrated hydrochloric acid, concentrated sulfuric acid, and hydrobromic acid.

16. The process according to claim 11, wherein the first mineral acid of (iv) is sulfuric acid.

17. The process according to claim 11, wherein the second mineral acid of (iv) is sulfuric acid.

18. The process according to claim 11, wherein the organic solvent of (i) is selected from the group consisting of tetrahydrofuran, 1,4-dioxane, toluene, benzene, acetonitrile, dimethyl formamide, dimethyl sulfoxide, ethyl acetate, and acetone.

19. The process according to claim 11, wherein the organic solvent of (i) is acetonitrile.

20. A process for synthesizing tilorone and tiolrone salts as an immunomodulator and a broad-spectrum antiviral agent, the process comprising:
(i) dissolving 2,7-dihydroxy-9-fluorenone prepared according to the process of claim 1 in an organic solvent to obtain a solution;
(ii) adding 2-bromo-N,N-diethylethylamine hydrobromide and a mineral alkali to the solution of (i) to obtain a mixture;
(iii) refluxing the mixture of (ii) at a reflux temperature from 75° C. to 160° C. to obtain tilorone of formula (f): 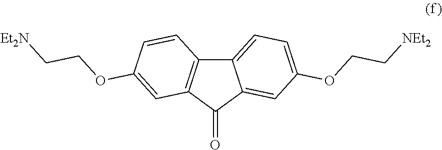
(iv) acidifying the tilorone of (iii) with an acid to afford a tilorone salt.

21. The process according to claim 20, wherein the reflux temperature is from 85° C. to 110° C.

22. The process according to claim 20, wherein the organic solvent is selected from the group consisting of tetrahydrofuran, 1,4-dioxane, toluene, benzene, acetonitrile, dimethyl formamide, dimethyl sulfoxide, ethyl acetate, and acetone.

23. The process according to claim 20, wherein the organic solvent is acetonitrile.

24. The process according to claim 20, wherein the mineral alkali is KOH.

25. The process according to claim 20, wherein the tilorone salt is selected from the group consisting of tilorone chloride, tilorone bromide, tilorone iodide, tilorone fluoride, tilorone citrate, tilorone oxalate, tilorone maleate, tilorone phosphate, tilorone tartrate, tilorone triflate, tilorone trifluoroacetate, and tilorone tetrafluoroborate.

26. The process according to claim 20, wherein the tilorone salt is tilorone dihydrochloride and is an orally active interferon inducing agent.
